(19) United States
(12) Patent Application Publication
(10) Pub. No.: US 2026/0335643 A1
(43) Pub. Date: Oct. 29, 2026

(54) MOTION SICKNESS PREVENTION AND MITIGATION ELECTRONIC DEVICE

(71) Applicant: NEURIVE CO., LTD., Gyeongsangnam-do (KR)

(72) Inventor: Ki Hwan HONG, Seoul (KR)

(21) Appl. No.: 19/115,398

(22) PCT Filed: Sep. 26, 2023
(86) PCT No.: PCT/KR2023/014821

§ 371 Date: Mar. 26, 2025

(30) Foreign Application Priority Data
Sep. 26, 2022 (KR) 10-2022-0121433
Sep. 26, 2023 (KR) 10-2023-0129117

(51) Int. Cl. A61N 1/36; A61F 7/00; A61N 1/04

(52) U.S. Cl. CPC A61N 1/36036; A61F 7/007; A61N 1/0456; A61N 1/36034; A61F 2007/0005

(57) ABSTRACT

Provided is a device which transmits stimulation in the form of an earset worn on wearer's ear to prevent and mitigate motion sickness of a user. The device comprises a plurality of electrodes which are in contact with different portions among the vagus nerve of the wearer and transmit electrical stimulation, and a processor for controlling the plurality of electrodes to control at least one among the frequency and strength of the stimulation. The device comprises a first set to come into contact with the left ear of a wearer and a second set to come into contact with the right ear of the wearer, wherein the same number of a plurality of electrodes are included in the first set and the second set, respectively, in a symmetric structure.

### TECHNICAL FIELD

The present disclosure relates to wellness or healthcare electronic devices, and more particularly, to electronic devices that prevent and mitigate sensory and nervous system-related body abnormalities such as motion sickness.

### BACKGROUND ART

Motion sickness is an abnormal symptom of nausea, vomiting, dizziness, sweating, and discomfort, which occur mainly in the vestibular organ when using a vehicle such as a car or a boat. Recently, motion sickness has also occurred during virtual reality (VR) experiences.

There are various descriptions for the cause of motion sickness, but some say this is a self-defense phenomenon that occurs when the body interprets a difference between vision and other senses as a danger signal. According to this opinion, in a situation where motion sickness is likely to occur, the autonomic nervous system determines that there is a problem in the body and makes the senses sensitive to find the cause. In particular, the sense of smell is greatly affected, which is said to be a remnant of the need to increase olfactory sensitivity when the cause of a physical abnormality was toxic food, such as spoiled food or poisonous mushrooms, from an evolutionary perspective.

It is effective to prevent motion sickness, and representative medicines for prevention are antihistamines, anticholinergics (parasympathetic blocking agents), spasmolytics (anticonvulsants), and sympathomimetic drugs.

The most commonly used antihistamines are first-generation antihistamines, such as meclizine, dimenhydrinate, and chlorpheniramine, and are mainly used as pyridoxine or a combination with caffeine. An anticholinergic is scopolamine, and although this is taken orally as a combination with an antihistamine, a patch is widely known. Since the medicinal effect of the patch lasts for 3 days, this may be a good choice when long-term motion sickness prevention is needed, but this is strong and toxic enough to paralyze the nerves, so care must be taken when handling this.

Drugs such as an antihistamine and scopolamine have anticholinergic side effects, such as blurred vision, dry mouth, constipation, and urine retention, and in some cases, motion sickness may become more severe due to the side effects. Children, pregnant women, and patients need to be more careful when using this, and motion sickness medications are difficult to use on a daily basis without risk.

Recently, a system that performs galvanic vestibular stimulation to reduce motion sickness in a cyber environment, such as VR, has been introduced, but this is a structure that is difficult to use universally and has a limitation such as being used for a special purpose in a special environment.

### DISCLOSURE OF THE INVENTION

### Technical Goals

An electronic motion sickness prevention and mitigation device that may be used safely instead of motion sickness medications with side effects caused by toxicity may be provided.

When electrical stimulation is transmitted to the body, a device with a fast and reliable effect on preventing and mitigating motion sickness by transmitting direct and appropriate stimulation to the vagus nerve may be provided.

Since motion sickness is mitigated in an appropriate method according to the situation experienced by a user, a device that is universal and capable of coping with various situations may be provided compared to previously known methods.

### Technical Solutions

According to an aspect, a device for preventing and mitigating motion sickness of a user by transmitting stimulation in the form of an earset worn on the ears of a wearer is provided. The device includes a plurality of electrodes configured to transmit electrical stimulation by contacting different portions of a vagus nerve of the wearer and a processor configured to control at least one of a frequency and intensity of stimulation by controlling the plurality of electrodes.

According to an embodiment, the device may include a first set configured to contact the left ear of the wearer and a second set configured to contact the right ear of the wearer, in which the first set and the second set may each include a plurality of electrodes of the same number in a symmetrical structure.

According to an embodiment, the plurality of electrodes may include at least two electrodes of 1) a first electrode inserted into an external auditory canal through a cavum concha of the wearer in an in-ear manner and configured to transmit electrical stimulation to the external auditory canal, 2) a second electrode configured to contact a cymba concha of the wearer, 3) a third electrode configured to contact a tragus of the wearer, and 4) a fourth electrode approaching around a rear portion of the ears of the wearer and configured to contact a mastoid process of the wearer. For example, but without being limited thereto, a group of the first to fourth electrodes is symmetrically disposed on each of the first set and the second set.

According to an embodiment, the device may further include a communicator communicatively connected to an external device and configured to receive external data. In addition, the processor may be configured to adaptively adjust an output of at least one of the plurality of electrodes according to the external data.

According to an embodiment, the electrical stimulation may be constant current stimulation in a predetermined size range, for example, in a range of 7 milliamperes (mA) or less. In addition, the frequency may be adjusted in a predetermined range of 30 hertz (Hz)

According to another embodiment, at least one of the plurality of electrodes may be configured to exchange thermal energy to cause a change in the temperature of at least one of three semicircular canals of the wearer. The exchange of thermal energy may cause a change in the temperature of the skin by an electrode in contact with the skin momentarily providing (donating) heat to a contact area or absorbing heat. In particular, such a change may occur differentially in the left and right ears, thereby inducing a change in sensation in the wearer.

According to another embodiment, the plurality of electrodes may further include a plurality of electroencephalogram (EEG) electrodes to measure an EEG of the wearer. In this case, the plurality of EEG electrodes may be equally disposed left and right in a symmetrical array form along a connector that connects the first set to the second set.

According to an embodiment, the processor may be configured to calculate a first acceleration vector a1 corresponding to a change in visual information of the wearer using measurement signals of the plurality of EEG electrodes and change a frequency or intensity of each of the plurality of electrodes so that a difference between the first acceleration vector a1 and a second acceleration vector a2, which is actually applied to the wearer and the device, is reduced.

In this case, the device may further include a sensor configured to measure the second acceleration vector a2.

In another embodiment, the second acceleration vector a2 may be obtained by information received from an external device that is communicatively connected to a device. For example, but without being limited thereto, the external device may be a vehicle, such as a car, a ship, or an airplane, and/or on-board diagnostics (OBD) connected to such a vehicle, a smartphone, a navigation system, a black box, or the like.

### EFFECTS OF THE INVENTION

According to embodiments, motion sickness may be effectively prevented and suppressed without the toxicity caused by existing motion sickness medications such as anticholinergics and antihistamines. Accordingly, this may be used for mitigating motion sickness by children, pregnant women, patients, and the elderly without worrying about drug addiction or side effects.

In addition, compared to the electronic motion sickness mitigation way through electrical stimulation or vibration, which is previously known, this directly stimulates the vagus nerve and generates an appropriate effect, so the effect of preventing and mitigating motion sickness is immediate and more certain.

In addition, this is universally applicable to various situations and environments, such as motion sickness caused by shaking while a user is on a vehicle, such as a car or a ship, virtual reality (VR) motion sickness caused by input visual information when the body of the user does not move, or the like, thereby having many application fields.

Furthermore, since this is expected to have a synesthetic effect by mutually combining a variety of sensory stimulation, such as electrical stimulation, acoustic stimulation, and temperature stimulation, to the vagus nerve there may be a synergy effect and it may be possible to adaptively respond to the cause of motion sickness.

It may be possible to adaptively offset the difference between a movement of a vehicle and an estimated movement according to visual processing in real time so that control suitable for a real situation of the user who is on the vehicle may be possible. Furthermore, the stability and balance of the autonomic nervous system through a variety of stimulation, such as sound, heat, and electricity, may improve the vehicle boarding satisfaction and health of the user. In addition, since connected control between biometric information and vehicle information is possible, the user may communicate with the state of the vehicle, thereby improving satisfaction such as safety, calling attention, and vehicle information sharing.

### BRIEF DESCRIPTION OF DRAWINGS

FIG. 1 is a block diagram illustrating a device according to an embodiment.

FIG. 2 is an example of an external view illustrating a structure of a device, according to an embodiment.

FIG. 3 is a diagram illustrating a structure of one side of the device according to an embodiment of FIG. 2 and positions of electrodes arranged thereon.

FIG. 4 is a diagram illustrating electroencephalogram (EEG) electrodes arranged in a device, according to an embodiment.

FIG. 5 is a block diagram illustrating a device according to another embodiment.

FIG. 6 is a view of a user wearing a device and boarding a vehicle, according to an embodiment.

FIG. 7 is a block diagram illustrating a device according to another embodiment.

### BEST MODE FOR CARRYING OUT THE INVENTION

Hereinafter, the embodiments will be described in detail with reference to the accompanying drawings. The scope of the right, however, should not be construed as limited to the embodiments set forth herein. In the drawings, like reference numerals are used for like elements.

The terms used herein are selected from terms generally understood by those skilled in the related art, but may have different meanings according to technical developments and/or changes, practices, and preferences of an engineer. Accordingly, the terms used herein should not be construed as limiting the technical spirit, and should be construed as illustrative terms to describe embodiments.

In addition, in a specific case, most appropriate terms are arbitrarily selected by the applicant. In this instance, the meanings of the arbitrarily used terms will be clearly explained in the corresponding description. Hence, the terms should be understood not by the simple names of the terms but by the meanings of the terms and the following overall description of this specification.

Devices 100, 200, 500, 600, and 700 according to embodiments may be electronic devices. As shown in the drawings, an electronic device may itself be in a form that may be operated independently through dedicated hardware and firmware. However, as one of ordinary skill in the electronics industry may easily predict, the devices 100, 200, 500, 600, and 700 may be equipped with hardware, such as electrodes and circuit boards, and may operate in conjunction with at least some external computing device (not shown), and even in this case, unless the spirit of the disclosure described according to embodiments is changed, it should be understood as belonging to the scope of rights.

For example, it may be possible that the illustrated devices are operated by receiving an electrical or electromagnetic signal from system-on-chip hardware and/or a computing device implemented by application software. Such a computing device may include, for example, at least one of a smartphone, a tablet personal computer (PC), a mobile phone, a video phone, an e-book reader, a desktop PC, a laptop PC, a netbook computer, a personal digital assistant (PDA), a portable multimedia player (PMP), an MP3 player, a mobile medical device, a camera, or a wearable device (e.g., a head-mounted device (HMD), such as electronic glasses, electronic clothing, an electronic bracelet, an electronic necklace, electronic appcessory, an electronic tattoo, a smartwatch), or an electrical system of a vehicle.

The electrical system of the vehicle may include, for example, at least one of an automotive infotainment device, electronic equipment for ships (e.g., navigation equipment for ships, gyrocompasses, or the like), avionics, a security device, and a vehicle head unit.

In addition, when the devices 100, 200, 500, 600, and 700 are specifically designed for the purpose of preventing and mitigating motion sickness in a virtual environment, the computing device may include at least one of furniture, a portion of a home infotainment system mounted in a building/structure, an electronic board, an electronic signature receiving device, a display device, or a projector. It is obvious to one of ordinary skill in the art that the devices according to embodiments may have their own shape modified or may be interoperated and/or operated by some external computing devices and may also have a form in which such a function is added while the main use of the devices is other than preventing motion sickness. Hereinafter, detailed embodiments are described based on these assumptions. It should be understood that the variations described above are possible even if not mentioned every time for each embodiment.

### Cause of Motion Sickness and Action of Autonomic Nervous System

Motion sickness is a phenomenon that occurs when the human body interprets the difference in vision and other senses as a danger signal. The autonomic nervous system, which determines that there is a problem with the body, tends to make other senses more sensitive to find the cause, and particularly, the sense of smell is greatly affected. The reason may be that this may be the influence of toxic foods such as spoiled foods or poisonous mushrooms.

The symptoms vary slightly from person to person, but people feel dizziness (vertigo), headache, and nausea (retching), which are usually caused by low blood pressure. In some cases, cold sweat flows, and unprovoked burping or yawning occurs. The sense of smell is strengthened, the stomach is weakened, the drowsiness is felt, and especially when retching worsens, the stomach acid comes out of the mouth and eventually causes vomiting, which spreads to the surroundings, causing other people who are also suffering from motion sickness to vomit as well, shocking and terrifying the surrounding people.

The symptoms last quite a long time even after getting out of a vehicle. The symptoms last at least 10 minutes, and a few hours on average. In a severe case, the symptoms may last more than two days.

It is known that the symptoms occur when the parasympathetic nerve (vagus nerve) connected to the stomach is overly excited, too much acetylcholine and histamine are released, and when these neurotransmitters stimulate again the vestibular organs, which in turn stimulates the vomiting center in the medulla oblongata. In the end, there may be various factors that cause motion sickness, but motion sickness may be understood as a result of the broken harmony and balance of the autonomic nervous system. According to embodiments, the motion sickness symptoms may not occur when mitigating and preventing these problems of the autonomic nervous system by electrically stimulating the vagus nerve.

### Prevention and Mitigation of Motion Sickness Through Vagus Nerve Stimulation

The vagus nerve is one of the cranial nerves and corresponds to the 10th cranial nerve. The vagus nerve originates from the brain, is distributed across the face, chest, and abdomen, and is involved in the control of the parasympathetic nerve that acts on the heart, lungs, and digestive tract as a mixed nerve including parasympathetic nerve fibers. The vagus nerve has the longest and most complex structure among 12 pairs of cranial nerves and includes both sensory fibers and motor nerve fibers. It is known that when electrically stimulating the vagus nerve, the cerebrum neural plasticity may be improved, the neural activity may increase, and the activity of the parasympathetic nerve among the activities of the autonomic nervous system may especially be controlled.

As the vagus nerve is stimulated by the device according to embodiments, the parasympathetic nerve is suppressed and the sympathetic nerve is activated. Accordingly, motion sickness is mitigated by the effect of a parasympathetic blocking agent (anticholinergic), spasmolytic (anticonvulsant), and sympathomimetic drug, similar to the mechanism of existing motion sickness medicine. As described above, the idea of mitigating motion sickness with low-frequency electrical stimulation or the like has been previously mentioned in several literatures, but this was unrelated to the vagus nerve or there were limitations such as not properly setting up stimulation for the vagus nerve, not being able to adaptively respond to the mechanism and situation that cause motion sickness, or the like, but the embodiments are differentiated in this respect. Detailed embodiments are described below with reference to the drawings.

### Configuration and Function of Motion Sickness Prevention Device

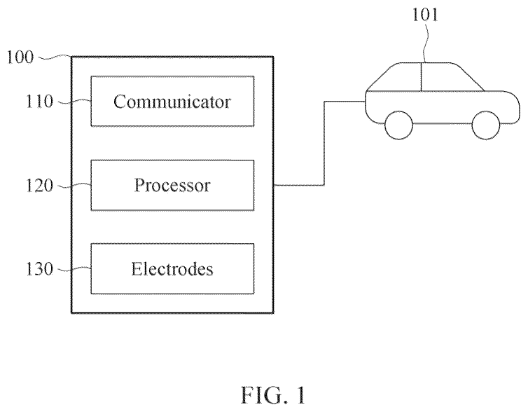

FIG. 1 is a block diagram illustrating the device 100 according to an embodiment.

The device 100 may prevent and mitigate motion sickness of a user by transmitting stimulation in the form of an earset worn on the ears of a wearer.

The device 100 according to an embodiment may include a plurality of electrodes 130 that transmits electrical stimulation by contacting the different portions of the vagus nerve of the wearer.

A processor 120 may control each of the plurality of electrodes and control at least one of the intensity and frequency of stimulation. According to an embodiment, the electrical stimulation may be constant current stimulation in a predetermined size range, for example, a range of 7 milliamperes (mA) or less. In addition, the frequency may be adjusted in a predetermined range of 30 hertz (Hz). However, the processor 120 may adjust the frequency as needed, and various cases requiring adjustment are described in detail with reference to each drawing.

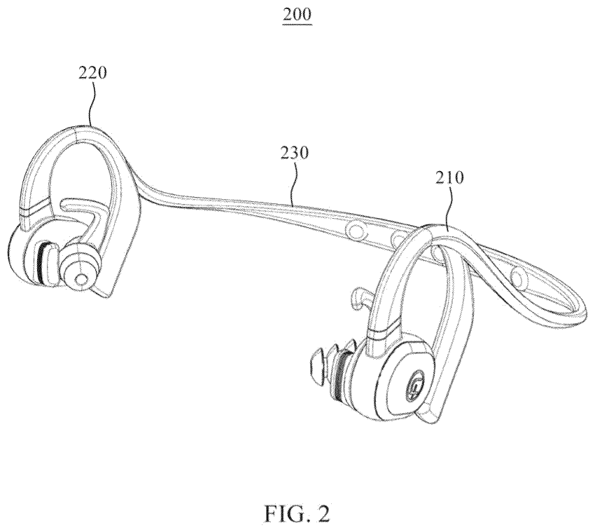

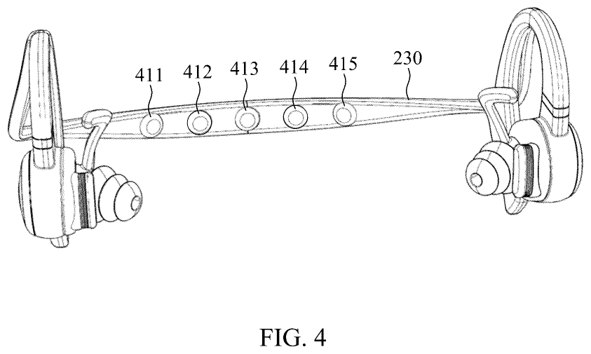

According to an embodiment, the device 100 may include a first set contacting the left ear of the wearer and a second set contacting the right ear of the wearer, in which the first set and the second set may each include a plurality of electrodes of the same number in a symmetrical structure, which structure is described in more detail below with reference to FIGS. 2 and 4.

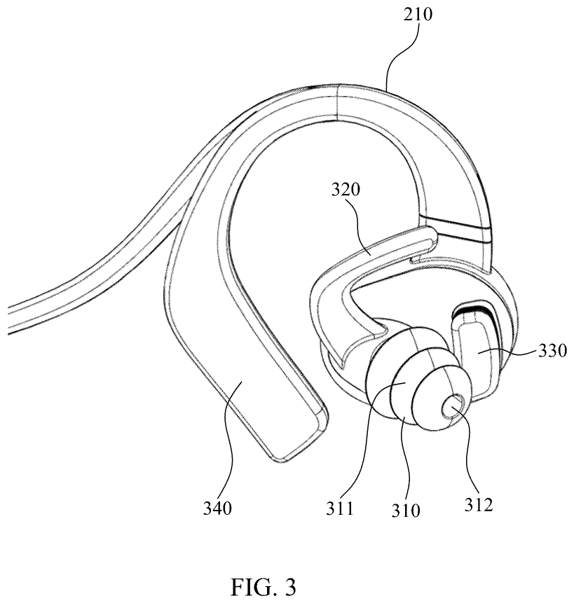

According to an embodiment, the plurality of electrodes may include at least two electrodes of 1) a first electrode inserted into the external auditory canal through the cavum concha of the wearer in an in-ear manner and transmitting electrical stimulation to the external auditory canal, 2) a second electrode contacting the cymba concha of the wearer, 3) a third electrode contacting the tragus of the wearer, and 4) a fourth electrode approaching around the rear portion of the ears of the wearer and contacting the mastoid process of the wearer. For example, but without being limited thereto, a group of the first to fourth electrodes may be symmetrically arranged on each of the first set and the second set. The structure and role of these electrodes are described in more detail below with reference to FIG. 3.

According to an embodiment, the device 100 may further include a communicator 110 communicatively connected to an external device 101 and receiving external data. In addition, the processor 120 may adaptively adjust an output of at least one of the plurality of electrodes according to the external data.

According to another embodiment, at least one of the plurality of electrodes 130 may exchange thermal energy to cause a change in the temperature of the three semicircular canals of the wearer. The exchange of thermal energy may cause a change in the temperature of the skin by an electrode in contact with the skin momentarily providing (donating) heat to the contact area or absorbing heat. In particular, such a change may occur differentially in the left ear and the right ear, thereby inducing a change in sensory of the wearer.

According to another embodiment, the device may further include a plurality of electroencephalogram (EEG) electrodes to measure an EEG of the wearer. In this case, the plurality of EEG electrodes may be equally disposed left and right in a symmetrical array form along a connector that connects the first set to the second set. The structure of the EEG electrodes is described in detail below with reference to FIG. 4.

According to an embodiment, the processor 120 may calculate first acceleration vector a1 corresponding to a change in visual information of the wearer using measurement signals of the plurality of EEG electrodes and may change the intensity or frequency of each of the plurality of electrodes 130 so that the difference between the first acceleration vector a1 and second acceleration vector a2, which is actually applied to the wearer wearing the device 100, is reduced. In some other embodiments, the second acceleration vector a2 may be obtained by information received from the external device 101. For example, but without being limited thereto, the external device 101 may be a vehicle, such as a car, a ship, or an airplane, and/or on-board diagnostics (OBD) connected to such a vehicle, a smartphone, a navigation system, a black box, or the like.

### Example of Structure of Motion Sickness Prevention Device

FIG. 2 is an example of an external view illustrating a structure of the device 200, according to an embodiment. In an embodiment, the overall shape of the device 200 is a structure in which a unit 210 on the left L and a unit 220 on the right R are held by a connector 230 and looks similar to neck band-type earphones. However, the units 210 and 220 may include a plurality of electrodes for stimulation of the vagus nerve, so there may be a difference in the structure and function from general earphones. To understand the structure of the electrodes in each unit, and the structure and arrangement of EEG electrodes that may be included in the connector, the description is provided with reference to FIGS. 3 and 4.

FIG. 3 is a diagram illustrating a structure of one side of the device according to an embodiment of FIG. 2 and positions of electrodes arranged thereon.

### Types and Roles of Electrodes

A first electrode 310 may be an electrode contacting the external auditory canal by being inserted into the external auditory canal through the cavum concha of the wearer in an in-ear manner. Since this is inserted into and contacts the external auditory canal located inside the ears, the first electrode 310 may protrude in the form of earbuds and have a plurality of protruding wings 311 to fix an electrode not to fall out easily while increasing the contact of the electrode.

When the first electrode 310 contacting the external auditory canal is made of a conductive polymer having flexibility and elastic restoring power, this may be appropriately deformed according to the size of the external auditory canal, which varies from person to person, while conducting electricity, and the adhesive force to the external auditory canal may increase. In addition, an electrode may be restored to its original shape and size after being taken out of the external auditory canal, and a problem due to deformation may not occur in the process of repeated use. A configuration that promotes sealing and internal adhesion using a polymer in an earphone or the like may be applied to the structure and shape of the first electrode 310 to which a conductive polymer is applied.

Additionally, the first electrode 310 to which a conductive polymer is applied may be configured to replace only the electrode by being manufactured in a detachable form, be replaced and used depending on a user by being manufactured in various sizes, and be used by replacing only the electrode when the electrode is damaged.

Furthermore, an acoustic unit (not shown) for generating sound may be provided in the device 200, sound or sound waves may be transmitted through a sound output passage 312 that is adjacent to the first electrode 310 inserted into the external auditory canal, and sound waves may be transmitted so that the user listens to music or sound for stabilizing the autonomic nervous system or listens to music or other sound sources that they like. This may induce meditation or mental stability, but there may be no need to limit the sound source to a certain sound source because the instability of the autonomic nervous system may be resolved while listening to the music that they like or watching videos that they are interested in, so it may be possible to listen to music provided by the device 200 or a sound source that they choose, depending on the selection of the wearer.

Through such sound stimulation, the activity of the cerebral cortex may be controlled, and this effect may affect nerve synapses related to degenerative brain diseases based on cerebrum neural plasticity, thereby promoting changes. Non-invasive vagus nerve stimulation according to the described embodiments may enhance neural plasticity of the cerebrum by stimulating the cerebral cortex through the nucleus tractus solitarius, the locus cceruleus, and the nucleus basalis, thereby promoting the formation of new synapses and improving training ability.

A second electrode 320 may be an electrode contacting the cymba concha of the wearer. The device 200 according to an embodiment may have electrodes contacting the external auditory canal and the cymba concha and transmitting electrical stimulation, and the second electrode 320 may contact the cymba concha and transmit electrical stimulation. Here, since the cymba concha of a human is concavely arranged above the crus helicis, the second electrode 320 may be in the form of a wing protruding along the shape of the cymba concha.

According to an embodiment, but without being limited thereto, the second electrode 320 contacting the cymba concha may be made of a wing-type conductive polymer having flexibility and elastic restoring force and may be appropriately deformed according to the structure and size of the cymba concha, which differ from person to person, and then inserted into the cymba concha, while conducting electricity, thereby increasing the adhesive force to the cymba concha due to the restoring force. The description related to the transmission of electrical stimulation is as mentioned above.

According to an embodiment, the device 200 may further include a third electrode 330 contacting the tragus. The third electrode 330 may have a structure in which the third electrode 330 naturally comes into close contact with the tragus surface by lightly pressing the tragus surface when the first electrode 310 is inserted into the external auditory canal by a predetermined degree or more. Since the size, shape, and location of the tragus are slightly different from person to person, the third electrode 330 to contact with the tragus surface may be molded to extend a little longer in the longitudinal direction of the face of the wearer to cover a wide range. The material may be selected from conductive materials including conductive polymers.

According to an embodiment, the device 200 may include a fourth electrode 340 that goes to the rear portion of the ears and contacts the mastoid process of the wearer. The fourth electrode 340 may also be made of a conductive polymer, such as conductive silicone or the like, and must contact the mastoid process well. Although the side of the unit 210 on the left is shown, referring to FIG. 2 together, as the elasticity of the connector 230 presses the fourth electrode, which is located each of the unit 210 on the left and the unit 220 on the right, toward the head of the wearer, the fourth electrode 340 may maintain the contact by slightly pressing the mastoid process. Furthermore, considering that this is a place where there is a lot of sweat, the surface of the fourth electrode 340 may be treated with a material capable of maintaining a contact point due to strong friction against The fourth electrode 340 contacts and electrically stimulates the mastoid process part so that the stability of the autonomic nervous system may increase through galvanic vestibular stimulation. According to an embodiment, the four electrodes of the first electrode 310 to the fourth electrode 340 may apply electrical stimulation to the body of the wearer simultaneously or alternately for each combination.

According to an embodiment, the stimulation current may be provided as constant current stimulation with the maximum intensity of 7 mA or less. As a result of the experiments conducted by the inventors, in the case of stimulation of about 30 Hz, it was found that the effect and safety of vagus nerve stimulation in a range smaller than 10 mA and the wearability of the user achieved the best compromise.

### Induction of Sense

Furthermore, according to another embodiment, at least a portion of the first electrode 310 to the fourth electrode 340, particularly the first electrode 310, may include a configuration that provides temperature stimulation to the contacting skin of the wearer. In otolaryngology, a caloric test is sometimes performed during a vestibular examination, which indicates that temperature stimulation induces a change in a vestibular response. The examination tests whether such a change in a vestibular response is normally felt as tilting or rotating, but the embodiment of the present disclosure may apply temperature stimulation for stabilizing the autonomic nervous system in addition to the electrical stimulation and sound stimulation described above. Artificially inducing the feeling of tilting or rotation through temperature stimulation may contribute to resolving the discrepancy in what the brain perceives both current visual information and sensory information. The embodiment of the present disclosure may use this to prevent or mitigate motion sickness.

### Active Movement Cancelling

According to an embodiment, the device 200 may sense brain waves of a human and roughly identify visual information viewed by a user. It is known in ‘Mayo Clinic's Galvanic Vestibular Stimulation (GVS) Technology’ that it is possible to determine whether the scenery being viewed is static or dynamic when looking ahead with both eyes by imaging brain waves or electrically observing the symmetry of brain activation areas. The problem is that the perception of the visual information perceived by the human body causes the brain to perceive movement in a certain direction, but in reality, the body does not move in that way, so there is a discrepancy with the perception of the brain by the sensation felt by the sensory system, such as the vestibular organ. In this case, a human may feel dizzy and motion sickness due to the discrepancy of information.

In an embodiment, it may possible to determine in which direction the user visually perceives a movement, that is, a rotational movement in the direction of roll, pitch, and yaw, a movement in the forward direction, a retreating backward movement, or a movement moving sideways to the left or right in the direction of view (when looking at an object through the side window while boarding on a moving car or when experiencing a similar experience through a virtual reality (VR) display). In addition, when such visual perception information is different from the actual movement acceleration of the body, a method of compensating for this (a method of artificially generating a sensation of compensation by inducing electrical stimulation or temperature differences and feeling the sensation, a method of adjusting the autonomic nervous system by at least temporarily increasing stimulation, or a method of preventing concentration on such visual dissonance through other stimulation, and a combination thereof) may be performed. In this specification, this may be referred to as active movement cancelling (AMC) or active acceleration cancelling (AAC) technology in the sense of offsetting dissonance with visual perception due to movement.

This may be a similar principle to active noise cancellation, which makes noise inaudible by causing destructive interference through sound waves opposing the surrounding noise. When the acceleration calculated/estimated by the movement, which is perceived from visual information so that the brain thinks that the body moves in that way and processes information, is vector a1, which makes the sensation corresponding to vector a2, which is the opposite vector, discrimination in electrical, sound, temperature (thermal) ways. In terms of sound, this may be similar to adjusting the balance of the front/rear/left/right speakers in a multi-channel speaker in real time and making it seem as if a visually visible object is passing by and making an actual sound. Even when electrically stimulating the vagus nerve, this balance difference may be generated to some extent by stimulating the front/rear/left/right differently. In addition, during the vestibular examination in otolaryngology, according to an embodiment, it may be possible to generate a sense of rotation through thermal differences, such as generating a feeling of rotation by bringing warm water into contact with one ear.

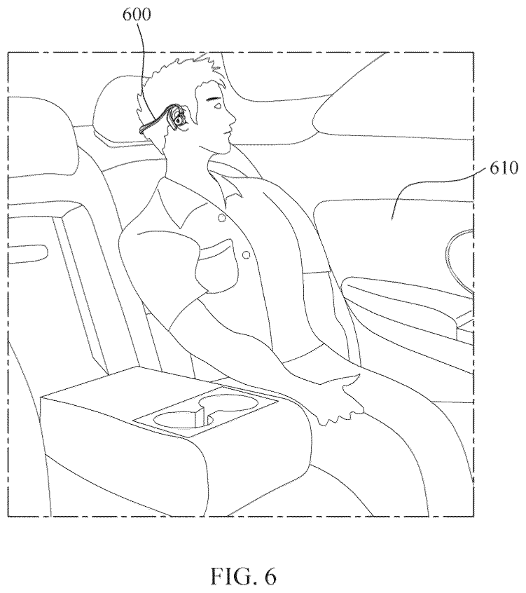

FIG. 4 is a diagram illustrating EEG electrodes 411 to 415 arranged in a device, according to an embodiment. As described above, to electrically visualize which part of the brain is activated and analyze the symmetry, the EEG electrodes 411 to 415 according to an embodiment may be arranged symmetrically left and right. A 5-axis or 7-axis gyro sensor and/or acceleration sensor may be included inside the connector 230. In such an embodiment, through the EEG electrodes 411 to 415, vector a1, which is visual perception acceleration estimated by brain activation, and vector a2, which is acceleration perceived by such a sensor, may be each calculated or estimated, and third vector a3 may be synthesized to minimize the difference between a1 and a2. Technically, the wearer mistakes sensory information that the vector a3 exists for stimulation such as electricity, sound, or heat. Accordingly, when the equation (a1=a2+a3) is established or substantially established, the cause of motion sickness, which is visual-cognitive dissonance, may be offset. AMC may also be used in FIG. 6.

### Connected Control Operated by Receiving Vehicle Information From External Device Such as Vehicle

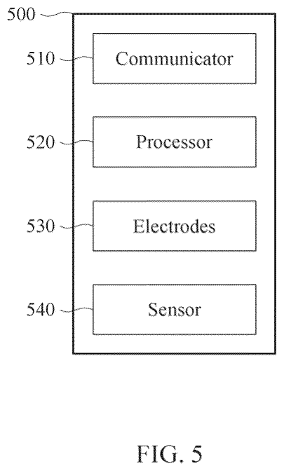

FIG. 5 is a block diagram illustrating a device according to another embodiment. A communicator 510 of the device 500 may receive movement information of a vehicle from an external device. The external device may be a vehicle itself. In this case, OBD data of the vehicle may be received in real time. In another embodiment, the movement information of the vehicle may be received from other electronic devices attached to the vehicle, such as a black box, a navigation device, or a smartphone of a user. It may be best to receive information including the direction and magnitude of acceleration according to the movement of the vehicle, especially rotational acceleration. However, it may also be possible to indirectly calculate acceleration by differentiating speed information and using steering information, operation information of accelerator pedals or brake pedals, or the like.

Instead of or in addition to such information from the vehicle, a sensor 540 mounted on the device 500 itself, such as a gyroscope sensor, may calculate the linear and/or rotational acceleration in real time, which is applied to the wearer of the device 500.

The processor 520 may then determine the degree of stimulation to be provided to the wearer through each of electrodes 530 for the above-described AMC or AAC and may provide a signal to the electrodes 530. The example of the structure of the electrodes 530 is as described above with reference to FIGS. 2 to 4.

Such a vehicle information interworking may be referred to as ‘connected control’ in this specification because this allows passengers to receive the movement information of the vehicle by themselves from the vehicle on which they board and use the movement information of the vehicle for generating stimulation to prevent and mitigate motion sickness, such as a recent connected drive such as Apple CarPlay or Android Auto.

FIG. 6 is a view of a user wearing the device 600 and boarding a vehicle, according to an embodiment. Motion sickness may be prevented or felt less through stimulation provided by the device 600 in a vehicle 610, such as electrical stimulation and sound stimulation. As described above, the device 600 may operate stand-alone but may be connected to other devices, such as the vehicle 610, to operate while receiving information according to the connected control described with reference to FIG. 5. For example, but without being limited thereto, such information transmission may be streamed in a multicast or broadcast manner, as established by the standard or industry standard. By using the devices 600 each of several passengers, motion sickness may be prevented while enjoying personalized stimulation to the extent suitable for one's setting. Additionally, in addition to preventing motion sickness, the connected control may also contribute to stabilizing the autonomic nervous system of a human by providing fun and distraction through additional elements such as controlling the vehicle's seat or playing music according to the weather.

Furthermore, since the device 600 includes an EEG sensor as described with reference to FIG. 4 or FIG. 5, information about the current state of a passenger, such as whether the passenger is sleeping, feeling anxious, dizzy, or excited, may be shared with the vehicle. A driver may then identify the information or may use the information to control the music or illumination of the vehicle. That is, the interworking of the device 600 with the vehicle 610 or other devices (not shown) connected to the vehicle by connected control technology may provide the possibility of various additional applications. In a broader sense, the interworking between providing biometric information of the passenger to the vehicle and providing the movement information of the vehicle or surrounding information to the passenger in real time may bring about an important change in the perspective of new industries and healthcare technologies.

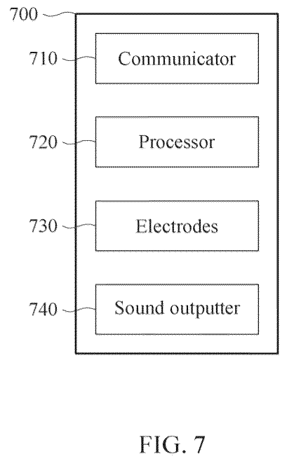

FIG. 7 is a block diagram illustrating the device 700 according to another embodiment. A processor 720 and electrodes 730 may be understood by referring to the various embodiments described above with reference to FIGS. 1 to 6. As briefly described with reference to FIG. 3, a sound outputter 740 may also provide various forms of sound stimulation to prevent and mitigate motion sickness of the wearer. For example, but without being limited thereto, the sound outputter 740 may transmit the sound or sound waves to the wearer through a sound output passage 312 that is adjacent to the first electrode 310 for external auditory canal insertion described with reference to FIG. 3.

The sound stimulation may be music or sound for stabilizing the autonomic nervous system or music or other sound sources that the user likes. Furthermore, in an important situation, this may be a guidance broadcast of a vehicle, which is received through a communicator 710 by the above-described connected control technology, information (e.g., “the vehicle will soon turn right,” “the vehicle will suddenly stop,” etc.) on the driving of the vehicle interworking with the navigation system, or the like. It may also be possible to output strong emergency alert information when a collision or accident is expected.

In contrast, rather than frequently transmitting information about the vehicle, a meditative sound and autonomous sensory meridian response (ASMR), which cognitively block out the vehicle and road condition, meditation music that may induce mental stability, or the like may be provided. In addition, depending on the selection of the wearer, the music that the wearer likes or the sound of a video that the wearer is interested in may be provided.

As described above, any music or any sound is not limited to some embodiments. The embodiment of the present disclosure describes that the user may mitigate and overcome the instability of the autonomic nervous system by receiving sound sources that are suitable for the user through the sound outputter 740.

It is known that the activity of the cerebral cortex is controlled through certain sound stimulation, and this effect may affect nerve synapses related to degenerative brain diseases based on the cerebrum neural plasticity, thereby promoting a change. Non-invasive vagus nerve stimulation according to embodiments may enhance the cerebrum neural plasticity by stimulating the cerebral cortex through the nucleus tractus solitarius, locus cceruleus, and nucleus basalis, thereby promoting the formation of new synapses and improving various cognitive abilities. A change in the cerebral synapses due to sound stimulation may induce the enhancement of cerebrum neural plasticity and synergy by non-invasive vagus nerve stimulation of the present disclosure, thereby enhancing the stabilizing effect of the autonomic nervous system.

The units described herein may be implemented using a hardware component, a software component and/or a combination thereof. A processing device may be implemented using one or more general-purpose or special purpose computers, such as, for example, a processor, a controller and an arithmetic logic unit (ALU), a DSP, a microcomputer, an FPGA, a programmable logic unit (PLU), a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is singular; however, one of ordinary skill in the art will appreciate that a processing device may include a plurality of processing elements and a plurality of types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or collectively instruct or configure the processing device to operate as desired. Software and/or data may be embodied permanently or temporarily in any type of machine, component, physical or virtual equipment, computer storage medium or device, or in a propagated signal wave capable of providing instructions or data to or being interpreted by the processing device. The software may also be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs or DVDs; magneto-optical media such as optical discs; and hardware devices that are specially configured to store and perform program instructions, such as read-only memory (ROM), RAM, flash memory, and the like. Examples of program instructions include both machine code, such as produced by a compiler, and files containing higher-level code that may be executed by the computer using an interpreter. The above-described devices may be configured to act as one or more software modules in order to perform the operations of the above-described embodiments, or vice versa.

A number of embodiments have been described above. Nevertheless, it should be understood that various modifications may be made to these embodiments. For example, suitable results may be achieved if the described techniques are performed in a different order and/or if components in a described system, architecture, device, or circuit are combined in a different manner and/or replaced or supplemented by other components or their equivalents.

Therefore, other implementations, other embodiments, and equivalents to the claims are also within the scope of the following claims.

1. A device for transmitting stimulation in a form of an ear set worn on ears of a wearer, the device comprising:
a plurality of electrodes configured to transmit electrical stimulation by contacting different portions of a vagus nerve of the wearer; and
a processor configured to control at least one of a frequency or intensity of stimulation by controlling the plurality of electrodes.

2. The device of claim 1, wherein the device comprises a first set configured to contact a left ear of the wearer and a second set configured to contact a right ear of the wearer, wherein the first set and the second set each comprise a plurality of electrodes of the same number in a symmetrical structure.

3. The device of claim 1, wherein the plurality of electrodes comprises at least two electrodes of:
1) a first electrode inserted into an external auditory canal through a cavum concha of the wearer in an in-ear manner and configured to transmit electrical stimulation to the external auditory canal;
2) a second electrode configured to contact a cymba concha of the wearer;
3) a third electrode configured to contact a tragus of the wearer; and
4) a fourth electrode approaching around a rear portion of the ears of the wearer and configured to contact a mastoid process of the wearer.

4. The device of claim 1, further comprising:
a communicator communicatively connected to an external device and configured to receive external data,
wherein the processor is configured to adaptively adjust an output of at least one of the plurality of electrodes according to the external data.

5. The device of claim 1, wherein the electrical stimulation is constant current stimulation in a predetermined size range.

6. The device of claim 3, wherein at least one of the plurality of electrodes is configured to exchange thermal energy to cause a change in a temperature of at least one of three semicircular canals of the wearer.

7. The device of claim 2, wherein the plurality of electrodes further comprises a plurality of electroencephalogram (EEG) electrodes to measure an EEG of the wearer,
wherein the plurality of EEG electrodes is arranged in a symmetrical array shape between the first set and the second set.

8. The device of claim 7, wherein the processor is configured to:
calculate a first acceleration vector a1corresponding to a change in visual information of the wearer using measurement signals of the plurality of EEG electrodes;
change a frequency or intensity of each of the plurality of electrodes so that a difference between the first acceleration vector a1 and a second acceleration vector a2, which is applied to the wearer and the device, is reduced.

9. The device of claim 8, further comprising:
a sensor configured to measure the second acceleration vector a2.

10. The device of claim 8, wherein the second acceleration vector a2 is obtained by information received from an external device that is communicatively connected to a device.
